(19) United States
(12) Patent Application Publication
(10) Pub. No.: US 2008/0275417 A1
(43) Pub. Date: Nov. 6, 2008

(54) Tampon with patterned end and method and apparatus for making same

(76) Inventors: Steven Ray Gilbert, Fairfield, OH (US); Jeanne Marie Hughes, Loveland, OH (US); Devin William Baldridge, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 11/799,914

(22) Filed: May 3, 2007

(51) Int. Cl. A61F 13/20

(52) U.S. Cl. 604/385.18

(57) ABSTRACT

A tampon comprising a pledget of absorbent material has a patterned impression in at least one end comprising one or more design elements. A method for making a tampon comprises compressing at least one end of a compressed pledget to form a patterned impression in the at least one end comprising one or more design elements is also provided. An apparatus for making a tampon with patterned impression in at least one end of a tampon is also disclosed.

### FIELD OF THE INVENTION

The invention relates to improved tampons and to apparatuses and methods of making such tampons.

### BACKGROUND OF THE INVENTION

Tampons are generally compressed absorbent structures typically shaped and sized to fit into a body cavity, such as, for example a human vagina. Often, due to the processes used for making tampons, the outer surface of the tampon, including the outer surface of the insertion end or withdrawal end, or both, may be relatively rough, uneven, crenulated, and unattractive. Such characteristics in the surface topography of a tampon may provide the user with a negative impression of the product, especially when seen prior to use. For example, the insertion end of a tampon is often visible to a user prior to application, such as, e.g., with a digital tampon and/or a tampon provided in an applicator that at least partially displays the outer surface of the tampon. Furthermore, surface topography of tampons, such as, e.g., the insertion end, may affect tampon performance, such as, for example, fluid absorption characteristics and/or user comfort during insertion.

Accordingly, it may be desirable to provide tampons that have a relatively appealing insertion end and/or withdrawal end prior to use. In addition, it may be advantageous to have a tampon with one or more ends with improved fluid absorption characteristics. Further, it may be desirable to provide a tampon that is more comfortable for the user to insert. Further still, it would be desirable to provide an apparatus and/or method of making a tampon that provides a tampon with a relatively appealing insertion end and/or withdrawal end, provides a tampon that is more comfortable for the user to insert, and/or provides the tampon with improved absorption properties during use.

### SUMMARY OF THE INVENTION

The present invention addresses one or more of the foregoing technical problems and provides a tampon which may comprise a compressed pledget of absorbent material. The tampon may have at least one end and a first patterned impression in the at least one end of the tampon comprising one or more design elements.

According to another aspect of this invention, an apparatus for making a tampon is provided and may comprise a compression machine for receiving and compressing an uncompressed pledget of absorbent material so as to form a pledget having at least one end and a header for compressing the at least one end of the compressed pledget and forming a patterned impression in the at least one end comprising one or more design elements.

According to yet another aspect of the invention, a method is provided for making a tampon which may comprise compressing an uncompressed pledget of absorbent material in a compression machine so as to form a compressed pledget having at least one end and compressing the at least one end of the compressed pledget with a header and thereby forming a patterned impression in the at least one end comprising one or more design elements.

Other features and advantages of the invention may be apparent from reading the following detailed description, drawings, and claims.

### BRIEF DESCRIPTION OF THE DRAWINGS

FIG. 1 is a side elevation view of a tampon made in accordance with an embodiment of the present invention.

FIG. 2 is a perspective view of an uncompressed pledget of absorbent material used to make a tampon such as that in FIG. 1.

FIG. 3 is a side elevation view of a tampon forming apparatus in accordance with an embodiment of the present invention.

FIG. 4 is a partial perspective view of the tampon forming apparatus illustrated in FIG. 3 with the compression machine in an uncompressed configuration.

FIG. 5 is a partial perspective view of the tampon forming machine illustrated in FIG. 3 with the compression machine in a compressed configuration.

FIG. 6 is a partial perspective view of the tampon forming apparatus illustrated in FIG. 3 with a compressed pledget in a mold cavity.

FIG. 7 is a perspective view of a tampon mold which forms part of the tampon forming apparatus illustrated in FIG. 3.

FIG. 8 is a partial sectional side elevation view of a header assembly which forms part of the tampon forming apparatus illustrated in FIG. 3.

FIG. 9 is a partial perspective view of the header apparatus illustrated in FIG. 8.

FIG. 10 is an end elevation view of the header which forms part of the header assembly illustrated in FIG. 8.

FIG. 11 is a partial sectional side elevation view of the tampon forming apparatus illustrated in FIG. 3 with a compressed pledget disposed in a compression cavity of the compression machine.

FIG. 12 is a partial sectional side elevation view of the tampon forming apparatus illustrated in FIG. 3 with a compressed pledget disposed partially in a mold cavity between a compression member and the header.

FIG. 13A is an end elevation view of a header comprising ring shaped die elements in accordance with an embodiment of this invention.

FIG. 13B is a perspective view of a tampon comprising an insertion end impressed with ring pattern elements corresponding to the ring die elements in FIG. 13A in accordance with an embodiment of this invention.

FIG. 14A is an end elevation view of a header comprising ring shaped die elements in accordance with an embodiment of this invention.

FIG. 14B is a perspective view of a tampon comprising an insertion end impressed with ring pattern elements corresponding to the ring die elements in the header of FIG. 14A in accordance with an embodiment of this invention.

FIG. 15A is an end elevation view of a header comprising star die elements in accordance with an embodiment of this invention.

FIG. 15B is a perspective view of a tampon comprising an insertion end impressed with star pattern elements corresponding to the star die elements in the header of FIG. 15A in accordance with an embodiment of this invention.

FIG. 16A is an end elevation view of a header comprising arcuate die elements in accordance with an embodiment of this invention.

FIG. 16B is a perspective view of a tampon comprising an insertion end impressed with arcuate pattern elements corresponding to the arcuate die elements in the header of FIG. 16A in accordance with an embodiment of this invention.

FIG. 17A is an end elevation view of a header comprising oval dimple die elements in accordance with an embodiment of this invention.

FIG. 17B is a perspective view of a tampon comprising an insertion end impressed with oval dimple pattern elements corresponding to the header in FIG. 17A in accordance with an embodiment of this invention.

FIG. 18A is an end elevation view of a header comprising cross-hatch die elements in accordance with an embodiment of this invention.

FIG. 18B is a perspective view of a tampon comprising an insertion end impressed with cross-hatch pattern elements corresponding to the cross-hatch die elements of the header of FIG. 18B in accordance with an embodiment of this invention.

FIG. 19A is an end elevation view of a header comprising concentric ring die elements in accordance with an embodiment of this invention.

FIG. 19B is a perspective view of a tampon comprising an insertion end impressed with concentric ring pattern elements corresponding to the concentric ring die elements of the header in FIG. 19A in accordance with an embodiment of this invention.

### DETAILED DESCRIPTION OF THE INVENTION

As summarized above, the present invention may encompass a tampon, an apparatus and method for making such a tampon. As will be explained in more detail below, tampons in accordance with embodiments of the invention may be made by compressing at least one end of a compressed pledget of absorbent material with a header to form a patterned impression in the at least one end. As a result, tampons made in accordance with certain embodiments of this invention may have at least one end with an appealing surface appearance, enhanced comfort for the user during insertion, and/or improved absorption properties during use.

Section A below describes terms for assisting the reader in understanding features of the invention, but not introducing limitations in the terms inconsistent with the context with which they are used in the specification. Section B describes tampons made in accordance with this invention. Section C is a detailed description of the drawings illustrating an apparatus in accordance with embodiments of this invention. Section D describes methods of manufacturing tampons in accordance with embodiments of this invention.

A. Terms

As used herein, “actuating” is any force delivered by an electric motor, mechanical transmission, pneumatically, linear drive, manual, and/or hydraulic.

As used herein, “cm” is centimeter, “g” is grams, “g/cc” and “g/cm³” are grams per cubic centimeter, “mm” is millimeters, and “psi” is pounds per square inch.

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

As used herein, the “compression member” is any member that can be used to compress a pledget. It can also function to transfer a compressed pledget.

The term “crease” as used herein, is the configuration of the compressed pledget that may be incidental or deliberate to compaction of the pledget. The creased configuration may be characterized by at least one bend at least in a portion of the pledget such that portion of the pledget may be positioned with a different plane than before with the observation that the surface regions near the bend may be in a different distal and angular relationship to each other after the folding has taken place. The term “crease” encompasses folds and wrinkles. In the case of the lateral compaction of a generally flat pledget, there may exist one or more creases in the form of bends or folds of generally 180 degrees such that the surface regions on either side of the bend may be juxtaposed or even in co-facial contact with each other.

As used herein, the term “density” is used with its common technical meaning with units of g/cm³ or g/cc. The density may refer specifically to that of a specific region or feature of the tampon as noted. The density will be measured, unless otherwise noted, by taking the weight divided by the geometric volume described by the shape. Unless noted, density refers to that of the overall structure and not the individual components, and will include in the measurement void volume of small pores and voids within the overall structure.

As used herein, “mold” refers to a structure for shaping a pledget during compression and/or retaining the shape for a compressed pledget subsequent to compression during the stabilization process. Molds have an inner surface defining an inner cavity and an outer surface. The inner cavity is structured to define or mirror the shape of the compressed absorbent pledget. Thus, in some embodiments, the pledget conforms to the shape of the inner cavity of the mold by a restraining force to result in a self-sustaining shape and is retained in the inner cavity during the stabilization process. In other embodiments, the mold retains the shape of the compressed pledget during the stabilization process. The inner cavity may be profiled to achieve any shape known in the art including, but not limited to, cylindrical, oval, rectangular, triangular, trapezoidal, semi-circular, hourglass, serpentine or other suitable shapes. The outer surface of the mold is the surface external to the inner surface and can be profiled or shaped in any manner, such as, rectangular, cylindrical or oblong. The mold may comprise one or more members. Suitable molds used in the present invention may include, but may not be limited to unitary molds, comprising one member, and split cavity molds. Examples of split cavity molds include those disclosed in U.S. patent application Ser. No. 10/150,050 entitled “Substantially Serpentine Shaped Tampon,” and U.S. patent application Ser. No. 10/150,055, entitled “Shaped Tampon,” both filed on Mar. 18, 2002.

As used herein the term “patterned impression” refers to a shape imparted to a surface by compressing material with a die. The shape imparted comprises one or more design elements corresponding to die elements of the die. Patterns may include a wide variety of design elements including but not limited to dimples, concentric circles, cross-hatching, stars, polygons, arcs, triangles, rings, lines, rectangles, petals, ellipsoids, tear drops, letters, or waves. In certain embodiments, the pattern can include design elements, such as, e.g., recesses and/or protrusions, that can be separated by land areas between the recesses and/or protrusions.

As used herein the term “pledget” refers to a construction of absorbent material prior to the compression of such construction into a tampon.

As used herein the term “rotary mold” refers to a mold for shaping a pledget during compression and/or retaining the shape for a compressed pledget subsequent to compression during the stabilization process comprising a rotary wheel that may have a plurality of mold cavities and may be configured for high volume production of tampons. The mold cavities may extend from an inlet side of the wheel to a discharge side of the wheel and each cavity may receive a compressed pledget from a compression machine as the wheel rotates. The rotary mold may also include a heating system such as steam heating for stabilizing the tampons and a discharge device for discharging finished tampons.

As used herein, “self-sustaining” is a measure of the degree or sufficiency to which the tampon retains its compressed form after stabilization such that in the subsequent absence of external forces, the resulting tampon will tend to retain its vaginally insertable shape and size. It will be understood by one of skill in the art that this self-sustaining form need not, and may not persist during actual use of the tampon. That is, once the tampon is inserted into the vagina or other body cavity and begins to acquire fluid, the tampon will begin to expand and may lose its self-sustaining form.

The term “shaped tampons,” as used herein, refers to compressed pledgets having either a substantially serpentine shape, an “undercut” or “waist,” or a non-uniform cross-section traversing from the insertion end to the withdrawal end of the tampon. The phrase “substantially serpentine” refers to a non-linear dimension between any two points spaced at least about 5 mm apart. The term “undercut” refers to tampons having a protuberance or indentation that impedes the withdrawal from a unitary mold. For example, shaped tampons may be hourglass shaped having at least one perimeter in the center of the tampon or “waist” that is less than both an insertion end perimeter and a withdrawal end perimeter.

As used herein, the term “split cavity mold” is a mold comprised of two or more members that when brought together complete the inner cavity of the mold. Each member of the split cavity mold comprises at least a portion of the inner surface that when brought together or closed completes the mold structure. The split cavity mold is designed such that at least two or more of the mold members can be at least partially separated, if not fully separated, typically after the tampon has acquired a self-sustaining shape, to expand the cavity volume circumscribed by the inner surface(s) thus permitting the easier removal of the tampon from the mold. Partial separation can occur when only a portion of two mold members are separated while other portions of the two mold members remain in contact. Where each member's inner surface portion joins the inner surface portion of another member, those points of adjacency can define a straight line, a curve, or another seam of any convoluted intersection or seam of any regular or irregular form. The elements of the split cavity in some embodiments may be held in appropriate position relative to each other by linking elements of any form including bars, rods, linked cams, chains, cables, wires, wedges, screws, etc.

The term “stabilized,” as used herein, refers to a tampon in a self-sustaining state wherein it has overcome the natural tendency to re-expand to the original size, shape and volume of the absorbent material and overwrap, which comprise the pledget.

As used herein, the term “tampon,” refers to any type of absorbent structure that is inserted into the vaginal canal or other body cavity for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as oval, rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is within a range from about 30 mm to about 60 mm in length. A tampon may be straight or non-linear in shape, such as curved along the longitudinal axis. A typical compressed tampon is within a range from about 8 mm to about 20 mm wide. The width of a tampon, unless otherwise stated in the specification, corresponds to the distance across the largest cross-section, along the length of the tampon and perpendicular to the longitudinal axis of the tampon.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

As used herein, the “tampon compression machine” is a machine assembly that includes parts that may compress a pledget. Typically a pledget compressed in the tampon compression machine is then transferred to a mold for final shaping into a self-sustaining form of a vaginally insertable shape where the mold may further compress parts of the pledget beyond that which the tampon compression machine accomplished prior.

B. Tampons

Tampons made according to certain embodiments of this invention may have a pattern impressed in at least one end which is visually appealing to the user, is more comfortable for the user to insert, and/or provides the tampon with improved absorption properties during use. According to certain embodiments, the tampon may have a patterned impression in the at least one end. The patterned impression can include one or more design elements. In certain embodiments, the one or more design elements can comprise recessed portions or protrusions or both and land areas between the recessed portions or protrusions or both.

According to certain embodiments, the tampon may extend from a withdrawal end to an insertion end distal from the withdrawal end and have a body portion extending between the withdrawal end and the insertion end. In such an embodiment, the tampon may have a patterned impression in the withdrawal end, the insertion end, or both. In addition, the body portion of the tampon may have substantially no patterned impression. According to certain embodiments, a tampon with a patterned end may have a body portion with creases formed during compression of the uncompressed pledget, but such creases may not be considered a patterned impression.

The patterned impression can be one or more colors. For example, the patterned impression can include one or more design elements and the one or more design elements can be the same or different colors. In certain embodiments, the patterned impression can include a first design element that is a first color and a second design element that is a second color. The first color and the second color can be different shades of a color, different colors, or any other suitable combination. In certain embodiments, the patterned impression can include one or more design elements that can be one or more colors that are contrasting to the color of the tampon, such as, the tampon insertion end and/or tampon body. In certain embodiments, the patterned impression includes recesses or protrusions and the recesses or protrusions, or both, are colored. Application of the color may be by any suitable means such as by spraying or dipping the respective tampon end with the dye or screen or transfer printing. According to certain embodiments, colorant may be applied to the tampon end by gaseous means such as steam used to stabilize the tampon end.

According to certain embodiments, the patterned impression may convey a message to the user. Messages may include, but are not limited to a directional symbol, such as a triangle or arrow indicating direction of insertion of the tampon, or words.

A tampon may, in certain embodiments, have a longitudinal axis extending from the withdrawal end to the at least one end. The at least one end may extend radially outwardly from the longitudinal axis further than does the body portion adjacent the at least one end. In certain embodiments, the insertion end is impressed with the patterned impression and forms a head. The head may be advantageous, for example, such that it may prevent the tampon from slipping back into an applicator during insertion of the tampon by a user.

In certain embodiments, a tampon may have a length and a patterned end that is 3 to 15 mm long. Further, in certain embodiments, a tampon may have a length and a patterned end that is no more than 50% of the length of the tampon.

According to certain embodiments, a tampon may have a patterned end with a surface area greater than the patterned end of the tampon would have without the patterned impression impressed in the end. This increased surface area may impart improved liquid handling properties, such as absorption properties, to the tampon insertion end or head. In certain embodiments, the patterned impression comprises recesses which can reduce the surface area of the patterned end that contacts the user's body during insertion and may thereby make the tampon more comfortable during insertion. In addition, the appearance of increased absorbency or comfort or both may be appealing to the user.

According to other embodiments, the insertion end of a tampon may have a density of at least about 0.3 g/cm³.

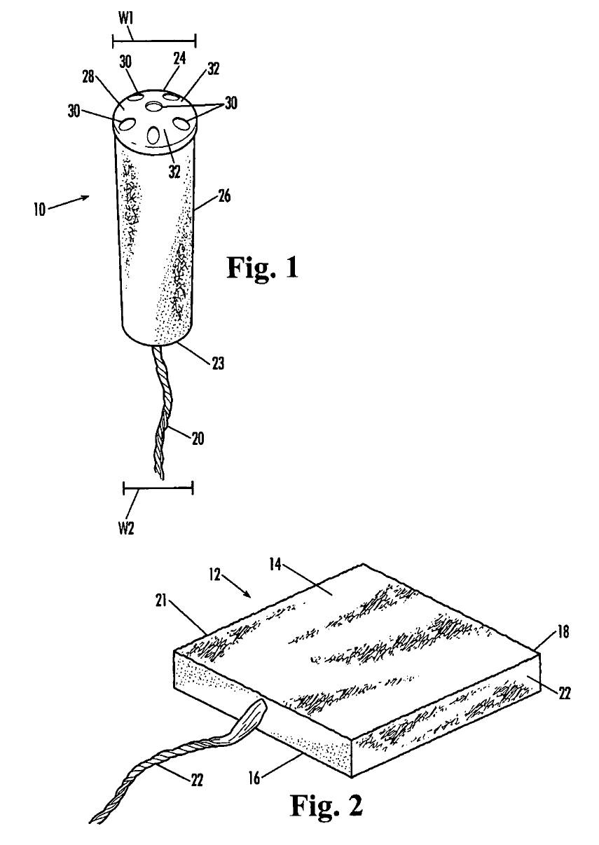

Turning to FIG. 1, a tampon 10 made in accordance with an embodiment of this invention is illustrated. Such an embodiment may be generally made by compressing an uncompressed pledget 12 of absorbent material 14, as illustrated in FIG. 2. The uncompressed pledget 12 extends from a withdrawal end 16 to an insertion end 18. A withdrawal cord or drawstring 20 may be connected to and extend from the withdrawal end 16 of the uncompressed pledget 12. Although the uncompressed pledget 12 is illustrated as having a square or rectangular shape, the uncompressed pledget 12 can have a variety of shapes including, but not limited to, oval, round, chevron, square, rectangular, and the like.

According to certain embodiments, the uncompressed pledget 12 has a length extending from the withdrawal end 16 to the insertion end 18 parallel to a longitudinal axis of the uncompressed pledget 12, a width extending substantially perpendicularly to the length of the uncompressed pledget 12 from a first side 21 to a second side 22, and a thickness extending substantially perpendicularly to both the length and width of the uncompressed pledget 12. In certain embodiments, the uncompressed pledget 12 is compressed with a compression having a major component in the widthwise direction; however, it should be understood that in certain other embodiments of this invention tampons can be made by compressing an uncompressed pledget with other types of compression, including but not limited to, radial compression and compression having a major component in a lengthwise direction.

The absorbent material 14 of the uncompressed pledget 12 may be constructed from a wide variety of liquid absorbing materials commonly used in absorbent articles. Such materials include but are not limited to rayon (such as GALAXY rayon, SARILLE L rayon both available from Kelheim Fibres GmbH of Kelheim, Germany), cotton, folded tissues, woven materials, nonwoven webs, synthetic and/or natural fibers or sheathing, comminuted wood pulp which is generally referred to as airfelt, or combinations of these materials. Other materials that may be incorporated into the pledget 12 include peat moss, absorbent foams (such as those disclosed in U.S. Pat. No. 3,994,298 issued to Desmarais on Nov. 30, 1976 and U.S. Pat. No. 5,795,921 issued to Dyer, et al.), capillary channel fibers (such as those disclosed in U.S. Pat. No. 5,356,405 issued to Thompson, et al. issued on Oct. 18, 1994), high capacity fibers (such as those disclosed U.S. Pat. No. 4,044,766 issued to Kaczmarck, et al. on Aug. 30, 1994), and super absorbent polymers or absorbent gelling materials (such as those disclosed in U.S. Pat. No. 5,830,543 issued to Miyake, et al. on Nov. 3, 1998). A more detailed description of liquid absorbing materials can be found in U.S. Pat. No. 6,740,070 to Raymond Agyapong.

The uncompressed pledget 12 may optionally include an overwrap comprising materials such as rayon, cotton, bicomponent fibers, polyethylene, polypropylene, other suitable natural or synthetic fibers known in the art, and mixtures thereof. In some embodiments, the uncompressed pledget 12 may have a nonwoven overwrap comprised of bicomponent fibers that have a polypropylene core surrounded by polyethylene manufactured by Vliesstoffwerke Christian Heinrich Sandler GmbH and Company KG (Schwarzenbach/Salle Germany) under the trade name SAS B31812000. In certain embodiments, the tampon may comprise a nonwoven overwrap of a hydro entangled blend of 50% rayon, 50% polyester available as BBA 140027 produced by BBA Corporation of South Carolina, US. In certain embodiments, the overwrap may be treated to hydrophilic, hydrophobic, wicking or nonwicking. In certain embodiments, the tampon may comprise an overwrap of formed film. The tampon, in certain embodiments, may include an overwrap covering the entire tampon or only a portion of the tampon.

The uncompressed pledget 12 may optionally include a secondary absorbent member, an additional overwrap, a skirt portion and/or an applicator. The withdrawal cord 20 attached to the uncompressed pledget 12 may be made of any suitable material in the prior art such as cotton and rayon. U.S. Pat. No. 6,258,075 issued to Taylor et al. describes a variety of secondary absorbent members for use in pledgets. An example of a skirt portion is disclosed in U.S. Pat. No. 6,840,927 to Margaret Hasse.

Thus, the tampon 10, as made in accordance with a certain embodiment of this invention and shown in FIG. 1, may extend from a withdrawal end 23 to an insertion end 24 distal from the withdrawal end 23 and have a body portion 26 extending between the withdrawal end 23 and the insertion end 24. The tampon 10 may have a longitudinal axis extending from the withdrawal end 23 to the insertion end 24. The insertion end 24 may have a surface 28 and a patterned impression of pattern elements in the insertion end 24 comprising recessed portions 30 and land areas 32 between the recessed portions 30. Further, the body portion 26 may have substantially no patterned impression, such as, e.g., as shown in FIG. 1. Although it is the insertion end 24 that is impressed with the patterned impression in the embodiment in FIG. 1, the withdrawal end 23 may also be impressed with the patterned impression, or both the withdrawal end 23 and the insertion end 24 may be impressed with the patterned impression. According to certain embodiments, the withdrawal end 23 could be formed so as to have the structures described herein with regard to the insertion end 24.

According to certain embodiments, the insertion end 24 of the tampon 10 may extend radially outwardly from the longitudinal axis further than does the body portion 26 adjacent the insertion end 24. This gives the insertion end 24 the shape of a head. In other words, the insertion end 24 of the tampon 10 may have a maximum width W1 greater than the width W2 of the body portion 26 of the tampon 10 adjacent the insertion end 24.

C. Tampon Manufacturing Apparatus

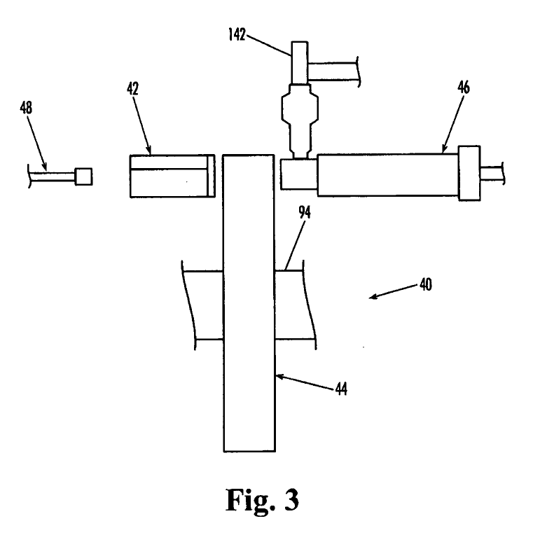

Turning to FIG. 3, a tampon forming apparatus 40, in accordance with certain embodiments of this invention, is illustrated. The tampon forming apparatus 40 may generally comprise a compression machine 42, a mold 44, and a header assembly 46. The mold 44 may be disposed between the compression machine 42 and the header assembly 46. The tampon forming apparatus 40 may also include a compression member 48.

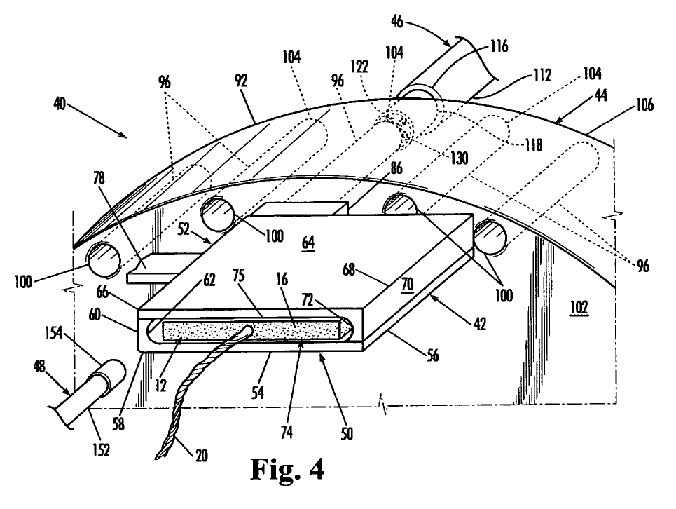

The compression machine 42, shown in FIGS. 4-6, may comprise a lower compression member 50 slidably engaged with an upper compression member 52. The lower compression member 50 may comprise a plate 54 extending from a first end 56 to a second end 58 and comprising an end wall 60 extending perpendicularly upwardly from the second end 58. The end wall 60 of the lower compression member 50 may have a concave inner surface 62. Likewise, the upper compression member 52 may comprise a plate 64 extending from a first end 66 to a second end 68 and may comprise an end wall 70 extending substantially perpendicularly downwardly from the second end 68. The end wall 70 may have a concave inner surface 72.

In FIG. 4, the compression machine 42 is in an exemplary uncompressed configuration and the lower compression member 50 may be slidably engaged with the upper compression member 52 such that the first end 66 of the upper compression member 52 is disposed adjacent to the end wall 60 of the lower compression member 50 and the end wall 70 of the upper compression member 52 is disposed adjacent to the plate 54 near the first end 56 of the lower compression member 50. In this uncompressed configuration, the lower compression member 50 and the upper compression member 52 may form a compression cavity 74 between the plate 54 of the lower compression member 50 and the plate 64 of the upper compression member 52 and between the concave inner surface 62 of the lower compression member 50 and the concave inner surface 72 of the upper compression member 52. The compression cavity 74 may extend from an inlet opening to a discharge opening. An actuating member 78 may extend from the upper member 52 for sliding the lower compression member 50 relative to the upper compression member 52.

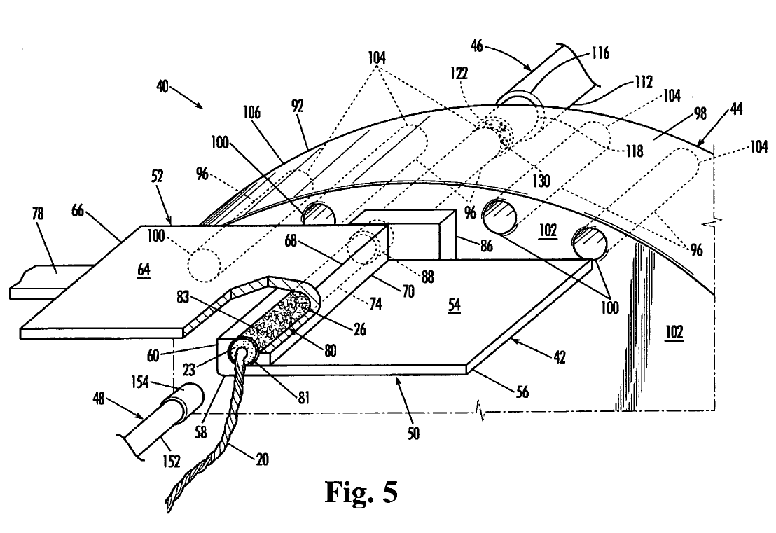

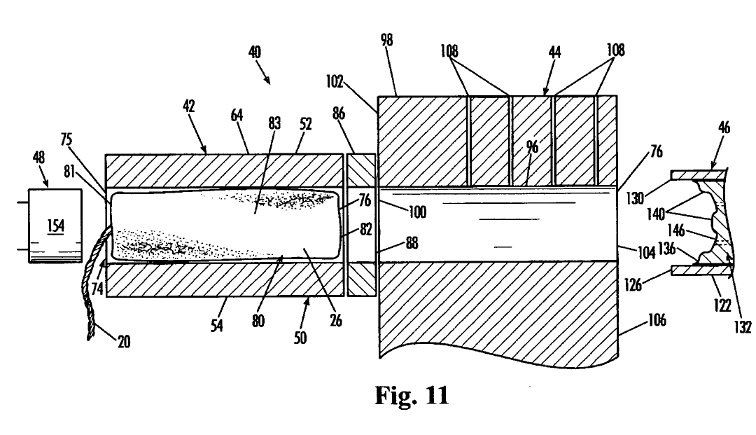

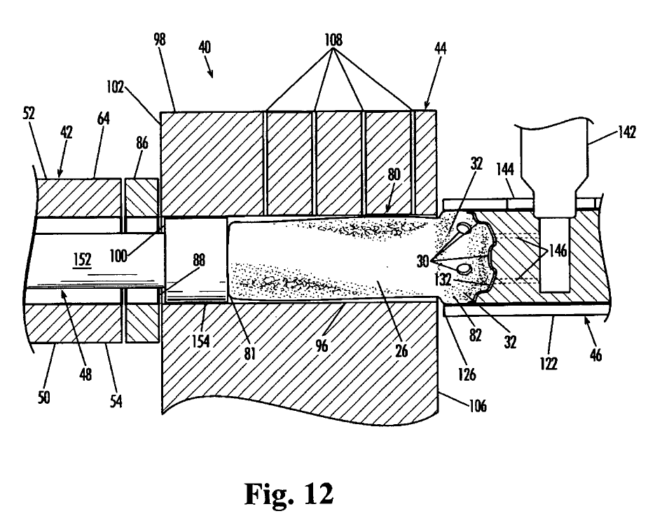

The compression cavity 74 of the compression machine 42 may be configured for receiving the uncompressed pledget 12 of absorbent material 14 in the uncompressed configuration of the compression machine 42 in FIG. 4. In certain embodiments, the lower compression member 50 and the upper compression member 52 may move relative to one another and thereby compress the uncompressed pledget 12 between the end walls 60 and 70. The compression machine 42 can compress the uncompressed pledget 12 to form a compressed pledget 80, as shown in FIG. 5. The compressed pledget 80 may extend from a withdrawal end 81 to an insertion end 82 (shown in FIG. 11) and may have a longitudinal axis extending from the withdrawal end 81 to the insertion end 82. The compressed pledget 80 may have a body portion 83 extending between the withdrawal end 81 and the insertion end 82.

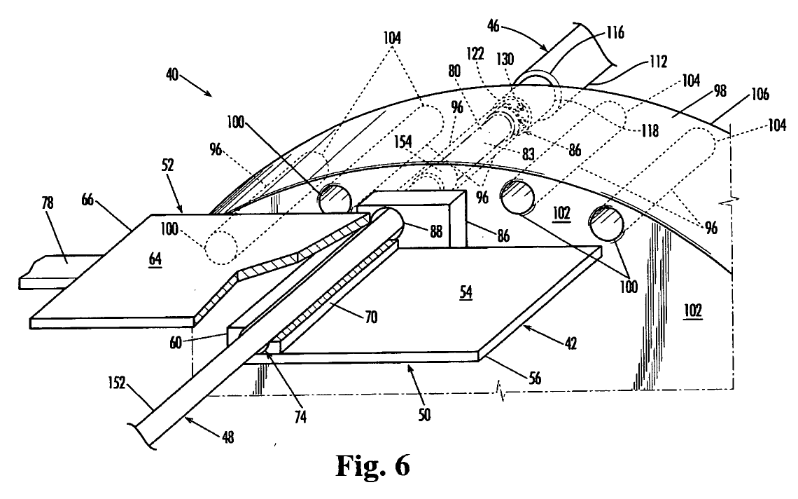

Other configurations for the compression machine 42 for carrying out the functions described herein are possible. The compression cavity 74 of the compression machine 42 may have a round cross-sectional shape as illustrated in FIGS. 5 and 6, but it should be understood that the compression machine cavity 74 may have other shapes as well including, but not limited to, round, square, triangular, and rectangular cross-sectional shapes.

As shown in FIGS. 4-6, the compression machine 42 may also include a stationary die 86 comprising a passage 88 extending through the stationary die 86. The stationary die 86 may be disposed between the compression cavity 74 of the compression machine 42 and the mold 44 such that the passage 88 in the stationary die 86 is aligned with the compression cavity 74 of the compression machine 42 when the compression machine 42 is in a compressed configuration as is illustrated in FIGS. 5 and 6.

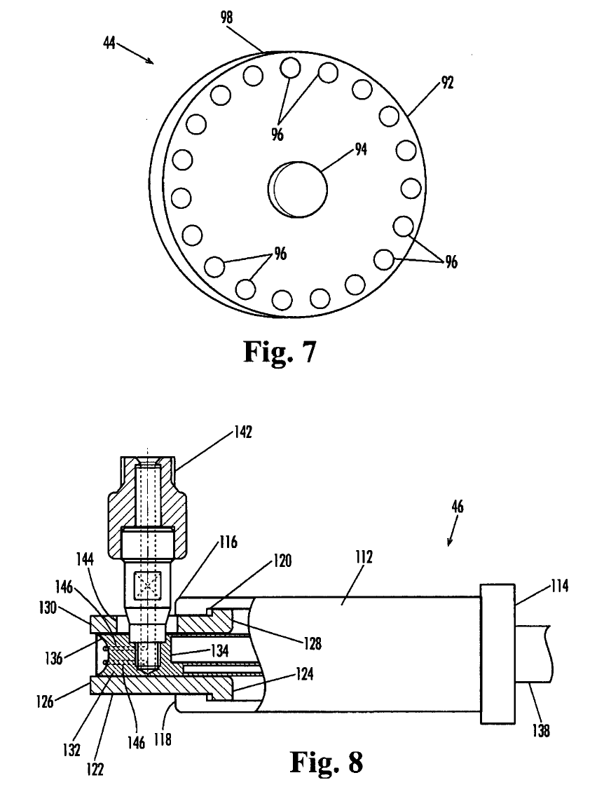

The mold 44 may be rotatably disposed adjacent the stationary die 86 of the compression machine 42. The mold 44 may comprise a wheel 92 rotatably mounted on a central axis 94, as shown in FIG. 7. A plurality of mold cavities 96 may be positioned about the periphery 98 of the wheel 92 and extend from respective inlet openings 100 in the inlet side 102 of the wheel 92 to respective discharge opening 104 in the discharge side 106 of the wheel 92 (shown in FIG. 11). Each mold cavity 96 has a longitudinal axis extending from the respective inlet opening 100 to the respective discharge opening 104 and has a width substantially perpendicular to the longitudinal axis. The compression machine 42 and stationary die 86 may be positioned in a stationary manner relative to the rotatable wheel 92 proximate the periphery 98 of the wheel 92 so that the compression cavity 74 of the compression machine 42 in the compressed configuration and the passage 88 in the stationary die 86 come into substantial alignment with the respective mold cavities 96 of the wheel 92 as the wheel 92 rotates the mold cavities 96 into position to receive compressed pledgets 80 from the compression member 42.

The mold 44 may have a plurality of steam passages 108 (shown in FIG. 11, but omitted from FIGS. 4-6 for clarity) extending from the periphery 98 of the wheel 92 to respective mold cavities 96 in the wheel 92 for delivering steam into the mold cavities 96 for stabilizing the compressed pledget 80 and forming the tampon 10. A source of steam and a steam delivery system for delivering steam to the steam passages 108 of the wheel 92 may be provided.

Although the mold 44 illustrated in FIG. 5 and other figures is a rotary mold, it should be understood that other molds may be suitable for certain embodiments of the invention. For example, single or multiple cavity stationary unitary molds may be used and split cavity molds may be used. Split cavity mold structures are disclosed in detail in U.S. Pat. No. 7,047,608 to Sagser and pending U.S. patent application Ser. No. 10/887,645 entitled “Compressed Gas Stabilized Tampon Having Multiple Folds”, filed on Jul. 9, 2005.

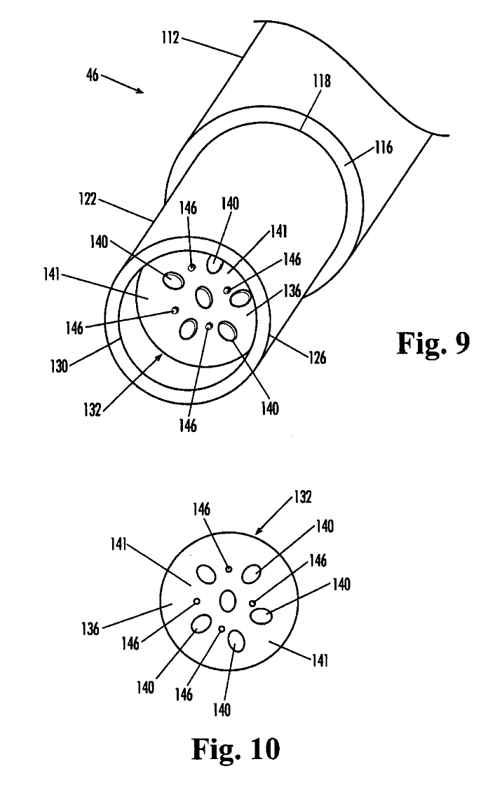

A header assembly 46 is illustrated in FIGS. 8 and 9 and may comprise a housing 112 extending from a rearward end 114 to a forward end 116. The forward end 116 of the tubular housing 112 may have an opening 118 defined by an annular shoulder 120.

The header assembly 46 may also comprise a sleeve 122 slidably disposed within the tubular housing 112 and extending from a rearward end 124 to a forward end 126. The sleeve 122 may comprise an annular flange 128 extending outwardly at the rearward end 124 of the sleeve 122. The annular flange 128 may be slidably disposed within the tubular housing 112 so that the annular flange 128 abuts the shoulder 120 of the tubular housing 112 when the sleeve 122 moves forward and limits forward movement of the sleeve 122 to prevent impact of the sleeve 122 against the discharge side 106 of the mold wheel 92. Thus, in certain embodiments, it may be desirable that, at its forward most position, the sleeve 122 of the header assembly 46 desirably does not make contact with the mold 44 so as to not damage either part. The sleeve 122 of the header assembly 46 may have an opening 130 in its forward end 126. The sleeve 22 may also have a longitudinal axis extending from the rearward end 124 to the forward end 126 and a width extending substantially perpendicularly to the longitudinal axis.

The header assembly 46 may also include a header 132 extending from a rearward end 134 to a pattern die 136. An actuating rod 138 may be connected to the rearward end 134 of the header 132 for actuating the header 132 within the sleeve 122. The patterned die 136 may have a variety of configurations. According to certain embodiments, the patterned die 136 may be concave or dome-shaped and may comprise a plurality of die elements 140 which may be protrusions or recesses separated by land areas 141. Although the die elements 140 illustrated in FIGS. 9 and 10 are oval in shape, in accordance with certain embodiments, the die elements of the pattern die may have a variety of shapes as will be illustrated further herein below.

According to certain embodiments, the header assembly 46 may be equipped with a steam valve 142 through a side opening 144 in the sleeve 122 for feeding steam from a steam source (not shown) through steam passages 146 extending through the header 132 to the patterned die 136.

According to certain embodiments, the width of opening 130 in the sleeve 122 of the header assembly 46 may be greater than the width of the mold cavity 96 and, as a result, the compressed pledget 80 may have an insertion end 82 shaped like a head which extends radially outwardly from the longitudinal axis of the compressed pledget 80 further than does the body portion 83 of the compressed pledget 80 adjacent the insertion end 82. Thus, the resulting tampon 10 may have an insertion end 24 shaped like a head which extends radially outwardly from the longitudinal axis further than does the body portion 26 of the tampon adjacent the insertion end 24. Such a tampon 10 can, for example, can be advantageous prevent the tampon 10 from slipping back into an applicator during insertion by a wearer.

The compression member 48 of the tampon forming apparatus 40 may comprise an actuating rod 152 and a head 154 attached to the actuating rod 152. Although not illustrated, in certain embodiments the head 154 could be shaped like the patterned die 136 of the header 132 for impressing a patterned impression in the withdrawal end 81 of the compressed pledget 80. In such an embodiment, the withdrawal end 81 and the insertion end 83 of the compressed pledget would be patterned. In accordance with certain embodiments, the uncompressed pledget 12 could be fed into the compression machine 42 in reverse, with the insertion end 18 proximate the inlet opening 75 of the compression cavity 74 and the withdrawal end 16 proximate the discharge opening 76, so that the withdrawal end 81 of the compressed pledget 80 is compressed by the header 132 and is impressed with the patterned impression.

The manner of actuation of the header assembly 46, and the sleeve 122 and header 132 within the header assembly 46, and the compression member 48 may be by any suitable means. In addition, according to certain embodiments, the tampon forming apparatus 40 may also include a device for removing finished tampons from the mold 44. Furthermore, means for rotating the mold wheel 92 may be by any suitable means. In accordance with certain embodiments, the rotation of the mold wheel 92 and actuation of the header assembly 46, and its component parts, as well as the compression member 48, may be synchronized.

A variety of materials may be used to make the components of the tampon forming apparatus 40. Suitable materials may be relatively rigid and include, but are not limited to, stainless steel, and in the case of microwave heat stabilization, microwave safe materials.

D. Method of Making Tampons

According to certain embodiments, the tampon 10 may be made by first inserting the uncompressed pledget 12 in the compression cavity 74 when the compression machine 42 is in the uncompressed configuration, as shown in FIG. 4.

The uncompressed pledget 12 may then be compressed in the compression cavity 74 by actuating, for example, the upper compression member 52 of the compression machine 42 and moving the end wall 70 of the upper compression member 52 toward the end wall 60 of the lower compression member 50 until a compression configuration, as illustrated in FIGS. 5 and 6 is reached. The amount of force required to compress the uncompressed pledget 12 may vary but suitable forces are typically from about 40 psi to about 300 psi. The degree of compression of the uncompressed pledget 12 in the compression machine 42 in a widthwise direction may be a major component of the compression. In accordance with certain embodiments of the invention, the major compression of the uncompressed pledget 12 in the compression cavity 74 in the widthwise direction is within a range from about 55% to about 90% of the original width of the uncompressed pledget 12. The degree of compression of the uncompressed pledget 12 in the thickness and lengthwise directions may be a minor component of the compression and, in accordance with certain embodiment of this invention, minor compression of the uncompressed pledget 12 in the compression machine cavity 90 in the thickness and lengthwise directions may be no more than about 40% of the original width of the uncompressed pledget 12. In accordance with certain embodiments of this invention, the major compression of the uncompressed pledget 12 in the compression cavity 74 in the widthwise direction may be from about 75% to about 85% of the original width of the uncompressed pledget 12 and the minor compression of the uncompressed pledget 12 in the compression cavity 74 in the thickness and lengthwise directions may be no more than about 30% of the original width of the uncompressed pledget 12.

After compression in the compression machine 42, the uncompressed peldget 12 may be formed into the compressed pledget 80. Further, the compressed pledget 80 may be ejected from the compression cavity 74, for example, by actuating the compression member 48 so that the compression member head 154 enters the inlet opening 75 of the compression cavity 74 and extends through the compression cavity 74 forcing the compressed pledget 80 through the passage 88 in the stationary die 86 and the inlet opening 100 of the mold cavity 96 aligned with the compression cavity 74 until the withdrawal end 81 of the compressed pledget 80 is within the mold cavity 96 and the insertion end 82 of the compressed pledget 80 extends out of the discharge opening 104 of the mold cavity 96. With the head 154 of the compression member 48 against the withdrawal end 81 of the compressed pledget 80, the sleeve 122 and header 132 of the header assembly 46 may be actuated toward the discharge opening 104 of the mold cavity 96 and the insertion end 82 of the compressed pledget 80. The shoulder 120 of the tubular housing 112 of the header assembly 46 may stop the forward motion of the sleeve 122 by catching the annular flange 128 of the sleeve 122 and prevents the forward end 126 of the sleeve 122 from impacting the discharge side 106 of the mold wheel 92. The forward end 116 of the sleeve 122, however, may substantially surround the insertion end 82 of the compressed pledget 80 and the header 132 may continue to move forward so that the patterned die 136 of the header 132 compresses the insertion end 82 of the compressed pledget 80 protruding from the mold cavity 96 and impart a head shape to the insertion end 82 of the compressed pledget 80 and impress recesses 30 into the insertion end 82 with the die elements 140 of the patterned die 136.

While the patterned die 136 of the header 132 is compressing the insertion end 82 of the compressed pledget 80, steam may be delivered by the steam valve 142 through steam passages 146 in the header 132 to the insertion end 82 to stabilize the insertion end 82 and self-sustain the insertion end 82 and recesses 30 impressed in the insertion end 82. Although the header 132 in the embodiment illustrated in FIG. 8 heats the insertion end 82 of the compressed pledget 80 with steam, other forms of stabilizing the insertion end 82 may be used. Such methods include, but are not limited to, thermal temperature gradient conduction or microwaving, as disclosed, for example, in U.S. Pat. No. 7,047,608.

After the insertion end 82 of the compressed pledget 80 is impressed and self-sustained by the header assembly 46, the header 132 may be withdrawn away from the mold 44 and the insertion end 82 of the compressed pledget 80 and the mold 44 may rotate and index until the next mold cavity 96 is aligned with the compression cavity 74 and the process of compressing and uncompressed pledget 12 to the compression cavity 44, compressing the uncompressed pledget 12 to form a compressed pledget 80, and feeding the compressed pledget 80 to the awaiting mold cavity 96 is repeated. Meanwhile, the already formed compressed pledget 80 with the patterned insertion end 82 may be carried by the rotatable mold 44 to a stabilization station at which steam may be delivered through the steam passages 108 into the body portion 83 of the compressed pledget 80 to stabilize and self-sustain the compressed pledget 80 to form the finished tampon 10. Again, methods of setting or stabilizing the tampon shape include, but are not limited to, heating the compressed pledget 80 with thermal temperature gradient conducting or microwaving, as well as steaming.

After the mold 44 rotates the finished tampon 10 from the stabilizing zone, the finished tampon 10 may be ejected from the mold cavity 96 by conventional means such as by pulling the withdrawal cord 20, manually or mechanically by grasping, hooking, picking, or clamping the tampon 10, or by vacuum withdrawal, other pneumatic means, or the like. Suitable means of tampon removal from a mold are described in U.S. Pat. No. 7,047,608.

As explained above, in certain embodiments, the compression member head 154 could be shaped like the patterned die 136 of the header 132 for impressing a patterned impression in the withdrawal end 81 of the compressed pledget 80. The withdrawal end 81 may then be impressed with the patterned impression using the compression member head 154 like a header. In such an embodiment, the withdrawal end 81 and the insertion end 83 of the compressed pledget would be patterned.

In accordance with certain embodiments, the uncompressed pledget 12 may be fed into the compression machine 42 in reverse, with the insertion end 18 proximate the inlet opening 75 of the compression cavity 74 and the withdrawal end 16 proximate the discharge opening 76, so that the withdrawal end 81 of the compressed pledget 80 is compressed by the header 132 and is impressed with the patterned impression.

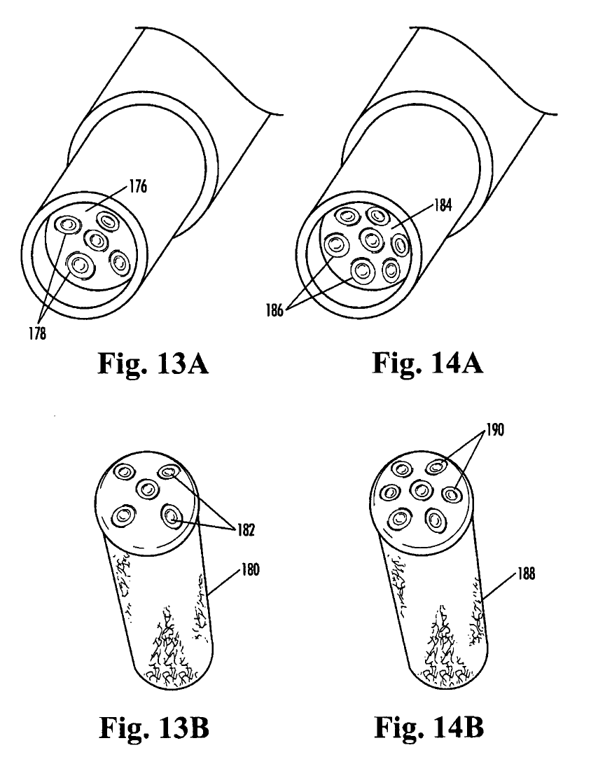

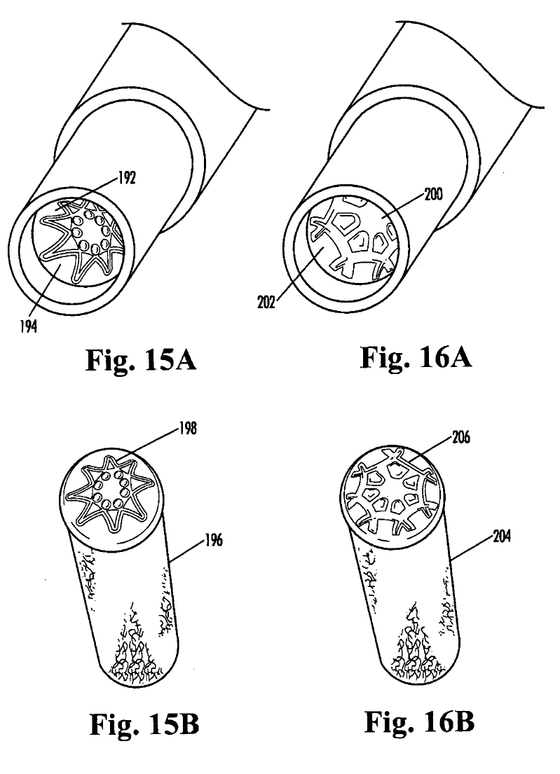

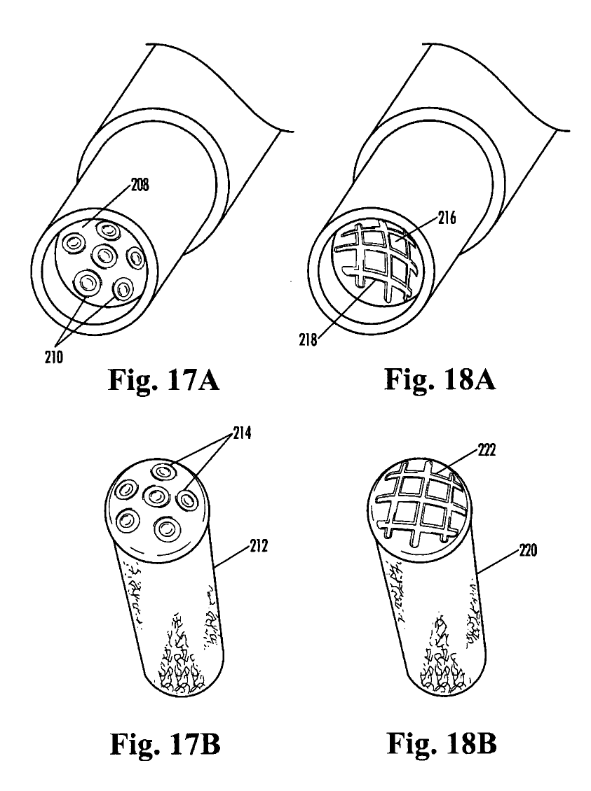

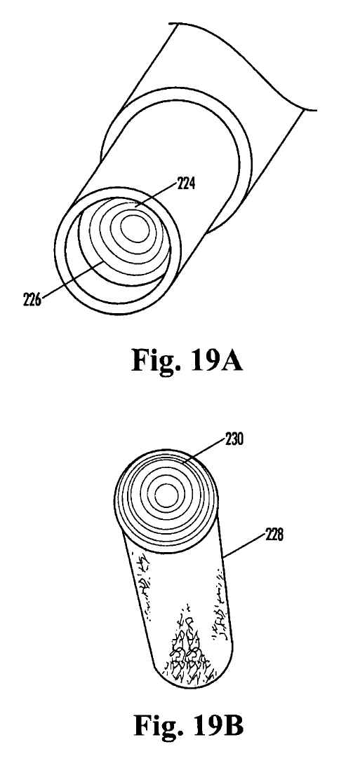

As illustrated in FIGS. 13A and 13B through 19A and 19B, the header assembly 46 can be modified to impart a variety of patterns to the insertion end 82 of the compressed pledget 80. The embodiment in FIG. 13A illustrates a header 176 which may comprise ring die elements 178 and the embodiment illustrated in FIG. 13B illustrates a tampon 180 which may comprise ring pattern elements 182 corresponding to the ring die elements 178 of the header 176. The embodiment illustrated in FIG. 14A illustrates a header 184 which may include ring die elements 186 and FIG. 14B illustrates an embodiment of a tampon 188 which may comprise ring pattern elements 190 corresponding to the ring die elements 186 in the header 184. FIG. 15A illustrates an embodiment in which a header 192 may comprise star die elements 194 and FIG. 15B illustrates an embodiment wherein a tampon 196 may be impressed with star pattern elements 198 corresponding to the star die elements in the header 192. An embodiment illustrated in FIG. 16A is a header 200 which may comprise arcuate die elements 202 and the embodiment illustrated in FIG. 16B is a tampon 204 which may be impressed with arcuate patterns 206 corresponding to the arcuate die elements 202. The embodiment illustrated in FIG. 17A is a header 208 which may comprise oval dimple die elements 210 and the embodiment illustrated in FIG. 17B is a tampon 212 having impressed therein oval dimple pattern elements 214 corresponding to the oval dimple die elements 210 in the header 208. The embodiment illustrated in the header 216 in FIG. 18A may comprise cross-hatch die elements 218 and the embodiments illustrated in FIG. 18B is a tampon 220 impressed with cross-hatch pattern elements 222 corresponding to the cross-hatch die elements 218 in header 216. The embodiment illustrated in FIG. 19A is a header 224 which may comprise concentric ring die elements 226 and the embodiment illustrated in FIG. 19B is a tampon 228 having impressed therein concentric ring patterns 230 corresponding to the concentric ring die elements 226 in header 224. According to certain embodiments, the compression member head 154 could be adapted to have the shapes of the headers shown in FIGS. 13A-19A for impressing a patterned impression in the withdrawal end 81 of the compressed pledget.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising a compressed pledget of absorbent material, the tampon having at least one end and a patterned impression in the at least one end comprising one or more design elements.

2. A tampon as in claim 1 wherein the one or more design elements include recessed portions or protrusions or a combination thereof and wherein the patterned impression includes land areas between the recessed portions or the protrusions or the combination thereof.

3. A tampon as in claim 1 wherein the tampon has a body and a longitudinal axis and the at least one end extends radially outwardly from the longitudinal axis further than does the body portion adjacent the at least one end.

4. A tampon as in claim 1 wherein the at least one end has a surface area greater with the patterned impression than the at least one end would have without the patterned impression impressed in the at least one end.

5. A tampon as in claim 1 wherein the at least one end has a density of at least about 0.3 g/cm³.

6. A tampon as in claim 1 wherein the one or more design elements are dimples, concentric circles, cross-hatching, stars, polygons, triangles, rings, lines, rectangles, petals, ellipsoids, tear drops, letters, or waves.

7. A tampon as in claim 1 wherein the tampon extends from a withdrawal end to an insertion end and the at least one end comprises the withdrawal end, the insertion end, or both the withdrawal end and the insertion end.

8. A tampon as in claim 1 wherein the tampon has a length and the at least one end extends no more than 50% of the length.

9. A tampon as in claim 1 wherein the one or more design elements are a color.

10. An apparatus for making a tampon comprising:
a compression machine for receiving and compressing an uncompressed pledget of absorbent material so as to form a compressed pledget having at least one end; and
a header for compressing the at least one end of the compressed pledget and forming a patterned impression in the at least one end comprising one or more design elements.

11. An apparatus of claim 10 wherein the one or more design elements include recessed portions or protrusions or a combination thereof and wherein the patterned impression includes land areas between the recessed portions or the protrusions or the combination thereof.

12. An apparatus as in claim 10 wherein:
the apparatus further comprises a tampon mold having a mold cavity for receiving the compressed pledget from the compression machine;
the apparatus further comprises a compression member for pushing the compressed pledget into the mold cavity;
the compressed pledget has a longitudinal axis and a body portion;
the mold cavity has a longitudinal axis extending from an inlet to an outlet and a width substantially perpendicular to the longitudinal axis of the mold cavity; and
the apparatus further comprises a header sleeve in which the header is movably disposed and the sleeve has a longitudinal axis and a width substantially perpendicular to the longitudinal axis of the header sleeve and greater than the mold cavity width such that, when the header compresses the at least one end, the sleeve receives the at least one end and the at least one end extends radially outwardly from the longitudinal axis of the compressed pledget further than does the body portion of the compressed pledget adjacent the at least one end.

13. An apparatus as in claim 10 wherein the header comprises a patterned die for imparting the patterned impression.

14. An apparatus as in claim 13 wherein the patterned die comprises one or more protrusions or one or more recesses or a combination thereof.

15. An apparatus as in claim 10 wherein the tampon mold is positioned between the compression machine and the header.

16. A method for making a tampon comprising:
compressing an uncompressed pledget of absorbent material in a compression machine so as to form a compressed pledget having at least one end; and
compressing the at least one end of the compressed pledget with a header and thereby forming a patterned impression in the at least one end comprising one or more design elements.

17. The method as in claim 16 wherein the one or more design elements include recessed portions or protrusions or a combination thereof and wherein the first patterned impression includes land areas between the recessed portions or the protrusions or the combination thereof.

18. The method as in claim 16 wherein the compressed pledget has a longitudinal axis and a body portion and the step of compressing the at least one end comprises forming the at least one end such that the at least one end extends radially outwardly from the longitudinal axis further than does the body portion adjacent the at least one end.

19. The method as in claim 16 wherein:
The method further comprises feeding the compressed pledget with a compression member into a tampon mold having a mold cavity for receiving the compressed pledget;
the compressed pledget has a longitudinal axis and a body portion;
the mold cavity has a longitudinal axis extending from an inlet to an outlet and a width substantially perpendicular to the longitudinal axis of the mold cavity;
the step of feeding the compressed pledget into the tampon mold cavity comprises pushing the compressed pledget until the at least one end extends outwardly beyond the tampon mold cavity;
the step of compressing the at least one end comprises substantially surrounding the at least one end extending outwardly beyond the tampon mold cavity with a header sleeve and moving the header through the sleeve to compress the at least one end, the header sleeve having a longitudinal axis and a width substantially perpendicular to the longitudinal axis of the header sleeve and greater than the mold cavity width such that when the header compresses the at least one end, the at least one end extends radially outwardly from the longitudinal axis of the compressed pledget further than does the body portion of the compressed pledget adjacent the at least one end.

20. The method as in claim 16 wherein the tampon extends from a withdrawal end to an insertion end and the at least one end comprises the withdrawal end, the insertion end, or both the withdrawal end and the insertion end.
